(19) United States
(12) Patent Application Publication
(10) Pub. No.: US 2026/0325164 A1
(43) Pub. Date: Oct. 22, 2026

(54) ATHERECTOMY DEVICES, SYSTEMS, AND METHODS

(71) Applicant: Bard Peripheral Vascular, Inc., Franklin Lakes, NJ (US)

(72) Inventors: Elizabeth M. Burkett, Scottsdale, AZ (US); Carter V. Righi, Mesa, AZ (US); Kyle A. Thomas, Phoenix, AZ (US); Marvin Clyde C. Vergara, Avondale, AZ (US); Zachary K. Willis, Mesa, AZ (US); Sarfaraz Imtiaz Shamji, San Antonio, TX (US)

(73) Assignee: Bard Peripheral Vascular, Inc., Franklin Lakes, NJ (US)

(21) Appl. No.: 19/108,926

(22) PCT Filed: Sep. 15, 2022
(86) PCT No.: PCT/US2022/076447

§ 371 Date: Mar. 5, 2025

(51) Int. Cl. A61B 17/3207; A61B 17/00

(52) U.S. Cl. CPC A61B 17/320758; A61B 2017/00685; A61B 2217/005; A61B 2217/007

(57) ABSTRACT

An atherectomy device is provided. The atherectomy device comprises an outer catheter body, an inner catheter body that extends through an outer catheter lumen of the outer catheter body, a front-facing cutting head having a portion rotatably connected to a distal end of the inner catheter body, and a rotatable screw member extending through an inner catheter lumen of the inner catheter body, wherein the rotatable screw member is configured to carry away material from the front-facing cutting head as the rotatable screw member rotates. A space between the outer catheter body and the inner catheter body defines a flushing lumen and the rotatable screw member having a guidewire lumen that extends coaxially with the outer catheter lumen and the inner catheter lumen.

### TECHNICAL FIELD

The present disclosure relates to atherectomy devices, systems, and methods for removal of materials from hollow bodies (e.g., blood vessels), and more particularly atherectomy devices, systems, and methods including a flushing lumen.

### BACKGROUND

Chronic Thromboembolic Pulmonary Hypertension (CTEPH) is a progressive form of pulmonary hypertension, where blood clots in the lungs are unresolved and a dense fibrous matrix of scar tissue develops around these clots. The resulting occlusion build up restricts the blood flow in the pulmonary arteries and pulmonary arterial pressure may increase, resulting in a pulmonary hypertension condition. It may therefore be useful to have devices providing intravascular removal of fibrous tissue from blood vessels. Currently this condition is addressed through a complex, expensive, and risky surgical procedure known as pulmonary endarterectomy or pulmonary thomboendarterectomy. The thromoendarerectomy procedure is an open surgery that requires a patient to be placed on coronary bypass and put into deep hypothermic circulatory arrest. Blood flow is started and stopped as surgeon cleans each vessel one at a time.

The devices of present disclosure may allow the patient to be placed under general anesthesia and blockages of blood vessels may be cleared intravascularly. The devices of present disclosure may also provide a minimally invasive solution to remove chronic occlusions and remove luminal patency or achieve luminal gain in the pulmonary arteries to address pulmonary hypertension.

### SUMMARY

To remove fibrous tissue from blood vessels, an atherectomy device may be used. Embodiments of the present disclosure are directed to atherectomy devices, systems, and methods for removal of materials from hollow bodies (e.g., blood vessels) with a front-facing cutting head as will be described in greater detail below.

In one embodiment, an atherectomy device is provided. The atherectomy device may include an outer catheter body, an inner catheter body that extends through an outer catheter lumen of the outer catheter body, a front-facing cutting head rotatably connected to a distal end of the inner catheter body, and a rotatable screw member extending through an inner catheter lumen of the inner catheter body. The rotatable screw member is configured to carry away material from the front-facing cutting head as the rotatable screw member rotates. A space between the outer catheter body and the inner catheter body defines a flushing lumen and the rotatable screw member having a guidewire lumen that extends coaxially with the outer catheter lumen and the inner catheter lumen.

In another embodiment, a system is provided. The system may include an atherectomy device. The atherectomy device may include an outer catheter body, an inner catheter body that extends through an outer catheter lumen of the outer catheter body, a front-facing cutting head rotatably connected to a distal end of the inner catheter body, and a rotatable screw member extending through an inner catheter lumen of the inner catheter body. The rotatable screw member is configured to carry away material from the front-facing cutting head as the rotatable screw member rotates. A space between the outer catheter body and the inner catheter body defines a flushing lumen and the rotatable screw member having a guidewire lumen that extends coaxially with the outer catheter lumen and the inner catheter lumen. The system may further include a driver coupled to the front-facing cutting head to rotate the front-facing cutting head.

In yet another embodiment, a method of using an atherectomy device is provided. The method may include advancing the atherectomy device. The atherectomy device may include an outer catheter body, an inner catheter body that extends through an outer catheter lumen of the outer catheter body, a front-facing cutting head rotatably connected to a distal end of the inner catheter body, and a rotatable screw member extending through an inner catheter lumen of the inner catheter body. The rotatable screw member is configured to carry away material from the front-facing cutting head as the rotatable screw member rotates. A space between the outer catheter body and the inner catheter body defines a flushing lumen and the rotatable screw member having a guidewire lumen that extends coaxially with the outer catheter lumen and the inner catheter lumen. The method may further include rotating the front-facing cutting head.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts one embodiment of an atherectomy device, wherein a front-facing cutting head and an inner catheter body are in a retracted position, according to one or more embodiments shown and described herein;

FIG. 2 schematically depicts the front-facing cutting head of FIG. 1, according to one or more embodiments shown and described herein;

FIG. 3A schematically depicts another embodiment of the front-facing cutting head having a helix shape, according to one or more embodiments shown and described herein;

FIG. 3B schematically depicts an isometric view of the front-facing cutting head of FIG. 3A, according to one or more embodiments shown and described herein;

FIG. 4A schematically depicts another embodiment of the front-facing cutting head including a peripheral wall, according to one or more embodiments shown and described herein;

FIG. 4B schematically depicts an isometric view of the front-facing cutting head of FIG. 4A, according to one or more embodiments shown and described herein;

FIG. 5A schematically depicts another embodiment of the front-facing cutting head including a non-helical blade being radially arranged, according to one or more embodiments shown and described herein;

FIG. 5B schematically depicts an isometric view of the front-facing cutting head of FIG. 5A, according to one or more embodiments shown and described herein;

FIG. 6A schematically depicts a cross sectional view of the atherectomy catheter of

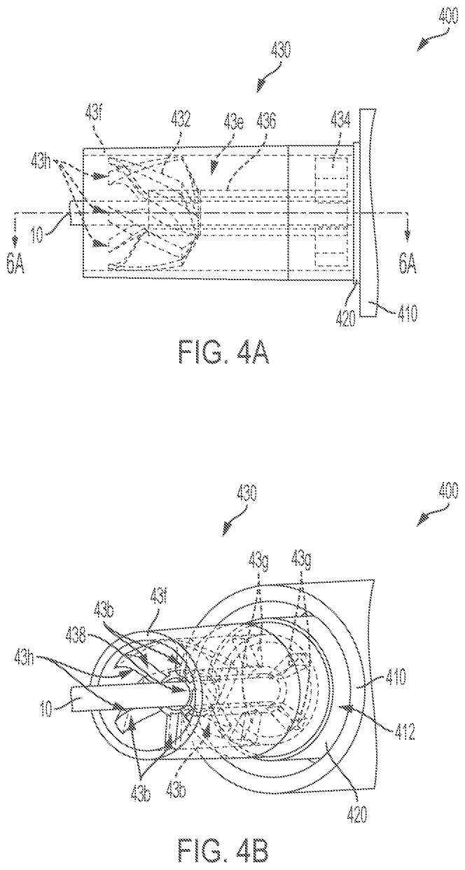

FIGS. 4A and 4B taken along section line 6A-6A, wherein the inner catheter body is in the retracted position, according to one or more embodiments shown and described herein; and

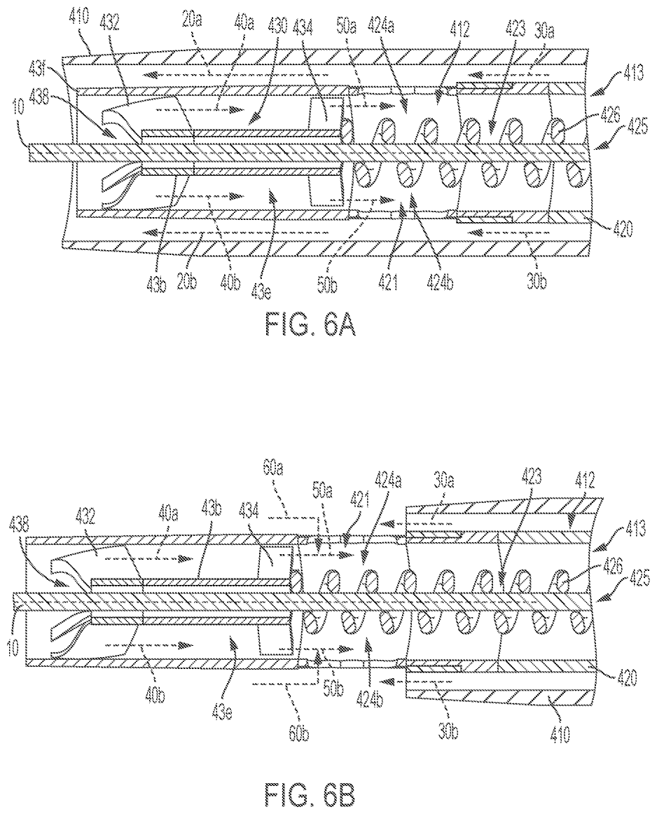

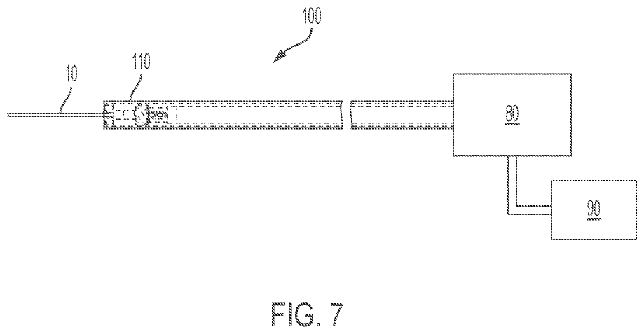

FIG. 6B schematically depicts a cross sectional view of the atherectomy catheter of

FIGS. 4A and 4B, wherein the inner catheter body is in an extended position, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments described herein are directed to atherectomy devices, systems, and methods for removal of materials from hollow bodies (e.g., blood vessels), and more particularly atherectomy devices, systems, and methods including a flushing lumen. For example, the atherectomy devices may be placed in a blood vessel or in an organ to remove materials such as blood clots or foreign materials therefrom. Over time, the blood clots may develop scar tissue around them and become fibrous tissue. As the occlusion grows and the blood vessels become narrower, blood pressure increases and may cause pulmonary hypertension. Pulmonary hypertension may affect arteries, heart, or the like. More specifically, CTEPH is a progressive form of pulmonary hypertension, where blood clots in the lungs are unresolved and a dense fibrous matrix of scar tissue develops around these clots. The resulting occlusion build up restricts the blood flow in the pulmonary arteries and pulmonary arterial pressure may increase, resulting in a pulmonary hypertension condition.

Embodiments of the present disclosure may provide intravascular removal of materials including fibrous tissue. For example, in some embodiments, an atherectomy device may include a front-facing cutting head to remove materials from blood vessels. These and additional features and benefits will be described in greater detail herein.

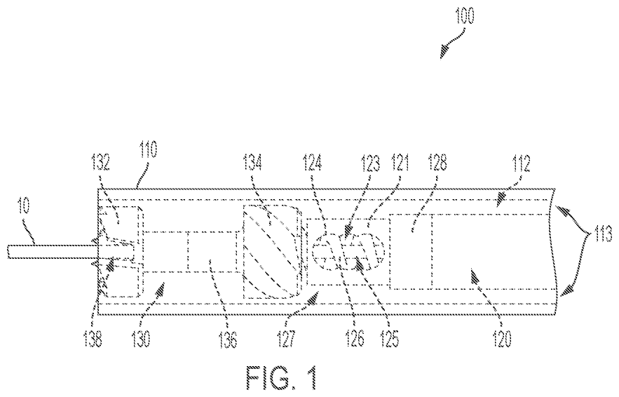

Referring to FIG. 1, an atherectomy device 100 for removing material from hollow bodies, is schematically depicted. The atherectomy device 100 may include an outer catheter body 110, an inner catheter body 120, and a front-facing cutting head 130. As used herein, the term “front-facing cutting head” refers to a cutting head that is configured to cut in an area primarily directly in front of the cutting head as opposed to an area surrounding an outer perimeter of the cutting head. The front-facing arrangement of the cutting head may be due to the shape of the cutting head and/or because the cutting head is surrounded by a tube or catheter body.

The inner catheter body 120 and the front-facing cutting head 130 may be disposed in an outer catheter lumen 112 when in a retracted position. The inner catheter body 120 may extend through the outer catheter lumen 112. A space between the outer catheter body 110 and the inner catheter body 120 may define a flushing lumen 113 where flushing fluid (e.g., saline, sterile saline, or other fluid) may be delivered there through. For example, a portion of the outer catheter lumen 112 may be defined as the flushing lumen 113. The flushing fluid may lubricate the front-facing cutting head 130 and may transfer heat due to friction between the rotatable screw member 126 and the inner catheter body 120. The flushing fluid may also remove heat generated by various components of the atherectomy device 100. The flushing fluid may irrigate hollow bodies during a procedure. Embodiments of the flushing lumen may allow the front-facing cutting head to run cooler with less blood flow, allowing the atherectomy device to be used in chronic lesion areas. The addition of the saline flush during aspiration may also reduce the amount of blood loss due to operation of the atherectomy device.

The front-facing cutting head 130 may be disposed at a distal end portion 121 of the inner catheter body 120. The front-facing cutting head 130 may include a distal cutting member 132, a proximal cutting member 134, and a shaft 136 that connects the distal cutting member 132 and the proximal cutting member 134. Each of the proximal cutting member 134 and the distal cutting member 132 may include one or more cutting portions (e.g., blades or edges). The proximal cutting member 134 and the distal cutting member 132 may have different configurations such as numbers, shapes, or edge shapes and may perform different functions selected from slicing and grinding. For example, the distal cutting member 132 may be configured for cutting and slicing fibrous tissue directly in front of the distal cutting member 132 and the proximal cutting member 134 may be configured for grinding the fibrous tissue already removed by the distal cutting member 132 as the fibrous tissue enters the catheter body 110. The front-facing cutting head 130 may have a guidewire lumen 138 extending coaxially with the outer catheter lumen 112 and the inner catheter lumen 123 (FIGS. 6A-6B). A guidewire 10 may be advanced through the guidewire lumen 138 so as to guide the atherectomy device 100 through hollow bodies. The front-facing cutting head 130 may be rotatably connected to the distal end portion 121 of the inner catheter body 120. The front-facing cutting head 130 may rotate with respect to the inner catheter body 120. The front-facing cutting head 130 may be rotated together with the inner catheter body 120.

The inner catheter body 120 may have a distal end portion 127 having one or more lateral openings 124. The lateral openings 124 may fluidly connect the inner catheter lumen 123 and the outer catheter lumen 112, which may create fluid communication between the outer catheter lumen 112 and the inner catheter lumen 123 via the lateral openings 124. The lateral openings 124 may extend in an axial direction of the inner catheter body 120. The lateral openings 124 may be formed having a shape of intersecting circular holes as depicted in FIG. 1. In alternative embodiments, the holes may overlap in the axial direction so as to form an axially extending opening. The holes may be separated from each other in the axial direction so as to form a plurality of lateral openings 124 spaced-apart in the axial direction. The lateral openings 124 may have a circular, rectangular, or any shape that may allow the fluid communication. Any suitable shape may be used for the lateral openings 124.

In embodiments including one lateral opening 124, the one lateral opening 124 may provide the fluid communication between the inner catheter lumen 123 and the outer catheter lumen 112. In embodiments including a plurality of lateral openings 124, the lateral openings 124 may be disposed facing each other through the inner catheter lumen 123, which may improve the fluid communication between the inner catheter lumen 123 and the outer catheter lumen 112 through the lateral openings 124. The plurality of lateral openings 124 configuration may provide increased fluid communication compared to one lateral opening 124. In embodiments, the distal end portion 127 of the inner catheter body 120 may be a component separately formed from the inner catheter body 120. In such case, the distal end portion 127 may be fixed to the inner catheter body 120. In other embodiments, the distal end portion 127 may be integrally formed with the inner catheter body 120, e.g., as a single extrusion. In embodiments, the lateral opening 124 may be used for lateral cutting. For example, a peripheral edge of the lateral opening 124 may be sharp enough to cut tissue or occlusions in the hollow bodies.

Still referring to FIG. 1, the atherectomy device 100 may include a rotatable screw member 126 disposed in the inner catheter body 120 and extending through the inner catheter lumen 123. The rotatable screw member 126 may be rotated to convey or transport materials removed from hollow bodies by the front-facing cutting head 130. For example, the front-facing cutting head 130 slices, chops, or cuts materials (e.g., fibrous tissue) in the hollow bodies into small pieces and the rotatable screw member 126 rotates to convey like screw conveyers. The rotatable screw member 126 may transport removed materials through the atherectomy device 100 and to an accumulation bag coupled to the atherectomy device 100. The rotatable screw member 126 may be rotated at a rate in excess of 40,000 revolution per minute (RPM). The rotatable screw member 126 may be an auger, continuous or non-continuous helix, a screw pump, or the like. The rotatable screw member 126 may rotate together with the front-facing cutting head 130, with respect to the inner catheter body 120. In this regard, the rotatable screw member 126 may transfer torque and rotational speed from a motor to the front-facing cutting head 130. The motor may be part of the atherectomy device 100 or be provided separate from the atherectomy device 100. In some embodiments, the rotatable screw member 126 may rotate separately from the front-facing cutting head 130. For example, the rotatable screw member 126 may be connected to a drive shaft that is not connected to the rotatable screw member 126.

The rotatable screw member 126 may have a guidewire lumen 125 that extends coaxially with the outer catheter lumen 112 and the inner catheter lumen 123. The guidewire 10 may be advanced through the guidewire lumen 125 to guide the atherectomy device 100 through hollow bodies. The rotatable screw member 126 may run over the guidewire 10. Removed materials may be conveyed in the axial direction of the inner catheter body 120 toward a proximal end of the inner catheter body 120. The removed materials may be collected into a reservoir coupled to or part of the atherectomy device 100. Negative pressure may be applied through the inner catheter lumen 123 to assist in aspirating removed material through the inner catheter lumen 123. The inner catheter lumen 123 may define an aspiration lumen where the removed material is carried away using the rotatable screw member 126.

In embodiments, the front-facing cutting head 130 may be coupled to the rotatable screw member 126 such that the front-facing cutting head 130 may rotate together with the rotatable screw member 126. In embodiments, the front-facing cutting head 130 and the rotatable screw member 126 may rotate together, while the inner catheter body 120 is stationary relative to outer catheter body 110.

In embodiments, one or both of the outer catheter body 110 and the inner catheter body 120 may be movable in the axial direction of the atherectomy device 100 relative to each other. The relative movements of the outer catheter body 110 and the inner catheter body 120 will be discussed later in detail with reference to FIGS. 6A-6B.

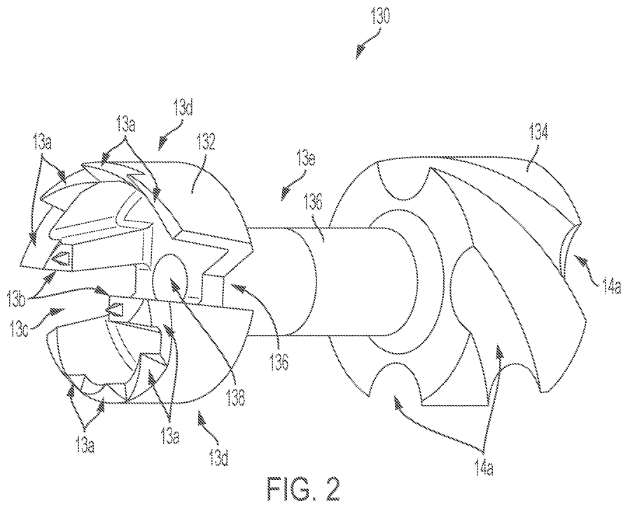

Referring to FIG. 2, the front-facing cutting head 130 is shown in isolation and may include the distal cutting member 132, the proximal cutting member 134, and the shaft 136 connecting the distal cutting member 132 and the proximal cutting member 134. The distal cutting member 132 may extend in a distal direction of the front-facing cutting head 130. Extended portions 13d may constitute a longitudinally extended wall of the distal cutting member 132. The extended portions 13d may form a peripheral wall of the distal cutting member 132. The peripheral wall may reduce possible unintended contact with hollow bodies (e.g., pulmonary vessel walls), and may protect hollow bodies from unintentional cutting. The extended portions 13d may have one or more disconnected portions 13c. The disconnected portions 13c may extend in the longitudinal direction and in the circumferential direction of the front-facing cutting head 130. The disconnected portions 13c may constitute notches that may allow fluid communication through the disconnected portions 13c and further through a space 13e formed between the distal cutting member 132, the proximal cutting member 134, and the shaft 136.

The shaft 136 may extend between the distal cutting member 132 and the proximal cutting member 134. The shaft 136 may be formed with one or more components that are coupled to each other to configure the shaft 136. The shaft 136 may extend continuously. The guidewire lumen 138 may extend through the shaft 136 to provide fluid communication therethrough.

The distal cutting member 132 may include circumferential cutting portions 13a and radial cutting portions 13b. The circumferential cutting portions 13a extend circumferentially along the periphery of the distal end portion of the front-facing cutting head 130. The circumferential cutting portions 13a ans the radial cutting portions 13b may provide different cutting properties such that to provide improved cutting performance. The circumferential cutting portions 13a may be serrated, scalloped, and the like. The radial cutting portions 13b may be located radially inward from the circumferential cutting portions 13a, toward the central axis of the front-facing cutting head 130. Each of the radial cutting portions 13b may include one or more protrusions. The protrusions may have a pyramid shape or any other shape that may have an apex and/or include a cutting edge facing toward the distal direction of the front-facing cutting head 130 that is configured for cutting an area in front of the front-facing cutting head 130. The combination of the circumferential cutting portions 13a and the radial cutting portions 13b with this particular embodiment may provide better performance with organized thrombus which characterizes the CTEPH condition by addressing different cutting areas that each of the circumferential cutting portions 13a and the radial cutting portions 13b provides.

The proximal cutting member 134 may have one or more groove portions 14a extending in the longitudinal direction of the front-facing cutting head 130. The groove portions 14a may be twisted or curved to form a thread like grooves along the outer surface of the proximal cutting member 134. The groove portions 14a may allow fluid communication there through. As a result, the disconnected portions 13c, the space 13e, and the groove portions 14a together may allow continuous fluid communication there through in the longitudinal direction of the front-facing cutting head 130.

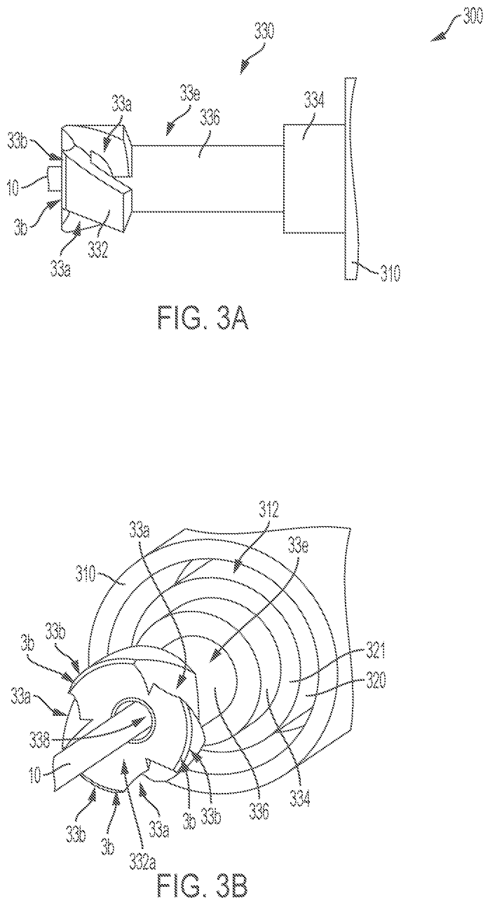

The front-facing cutting head may have any suitable configuration. FIGS. 3A and 3B illustrate another embodiments of an atherectomy device 300 with a front-facing cutting head 330. Referring to FIG. 3A, the front-facing cutting head 330 is in an extended position where the front-facing cutting head 330 extends out from an outer catheter body 310 and/or a tube (also represented by element 310). When placed the extended position, the front-facing cutting head 330 is uncovered by the outer catheter body 310 and exposed for a cutting operation. The front-facing cutting head 330 may have a distal cutting member 332, a proximal portion 334, and a shaft 336 connecting the distal cutting member 332 and the proximal portion 334. Cutting edges 3b extend distally out from a front face 332a of the distal cutting member 332. With the front facing cutting head 330, the atherectomy device 300 may exert power on the face of an organized thrombus and begin breaking up the tissue.

Referring to FIG. 3B, the front-facing cutting head 330 may have a guidewire lumen 338 that extends coaxially with an outer catheter lumen 312 and an inner catheter lumen (not shown due to obstruction by other components; equivalent to the inner catheter lumen 123 in FIG. 1). The distal cutting member 332 may extend in a distal direction of the front-facing cutting head 330. The distal cutting member 332 may include one or more cutting portions 33b. The distal cutting member 332 may have circumferential cutting portions 33b circumferentially disposed along the periphery of the distal end portion of the distal cutting member 332. The circumferential cutting portions 33b may have cutting edges 3b with a bevel facing away from the axis of the front-facing cutting head 330 in order to reduce contact between the cutting edges 3b and the vessel wall. The proximal portion 334 may be coupled to a distal end portion 321 of the inner catheter body 320. The front-facing cutting head 330 may rotate relative to or together with the inner catheter body 320. For example, the inner catheter body 320 may be a drive shaft that imparts torque and rotational speed to the front-facing cutting head 330. The inner catheter body 320 may also be used to move the front-facing cutting head 330 in the longitudinal direction relative to the outer catheter body 310 between the extended and retracted positions.

The distal cutting member 332 may have groove portions 33a that extend in the longitudinal direction of the front-facing cutting head 330. The groove portions 33a may be twisted or curved to form a thread or a helical like grooves along the outer surface of the distal cutting member 332. The groove portions 33a may allow fluid communication there through, which may be further fluidly connected to a space 33e formed between the distal cutting member 332, the shaft 336, and the proximal portion 334. The fluid communication may further connected to an outer catheter lumen 312 and/or to the inner catheter lumen. The space 33e may further be in fluid communication with the outer catheter lumen 312 and/or the inner catheter lumen. The outer catheter lumen 312 is disposed between the outer catheter body 310 and the inner catheter body 320.

FIGS. 4A and 4B illustrate embodiments of an atherectomy device 400 including a front-facing cutting head 430 with a peripheral wall 43f forming a tube (also represented by element 43f). Referring to FIG. 4A, the front-facing cutting head 430 is in an extended position where the front-facing cutting head 430 is located at least partially distally outside an outer catheter body 410. In embodiments, the outer catheter body 410 may be tapered toward a distal end of the outer catheter body 410. When in the extended position, the front-facing cutting head 430 and peripheral wall 43f are uncovered by the outer catheter body 410. The front-facing cutting head 430 may have a distal cutting member 432, a proximal cutting member 434, and a shaft 436 connecting the distal cutting member 432 and the proximal cutting member 434. The front-facing cutting head 430 may have the peripheral wall 43f which may form a tube that radially surrounds one or all of the distal cutting member 432, the proximal cutting member 434, and the shaft 436. In embodiments, the peripheral wall 43f may be tapered toward a distal end of the front-facing cutting head 430. The peripheral wall 43f may provide a shielded arrangement in sensitive anatomical geometries as the cutting surfaces of the distal cutting member 432 are partially shielded by the peripheral wall 43f. There is less likelihood of inadvertent contact with vessel walls in this configuration.

The specific arrangement of the blades in this embodiment are to aid in tissue removal and aspiration through the front of the cutting head and into the screw path.

The peripheral wall 43f may be integrally formed with the distal cutting member 432 and/or the proximal cutting member 434 or may be separately formed but fixed to the distal cutting member 432 and/or the proximal cutting member 434. The peripheral wall 43f may rotate together with the distal cutting member 432 and/or the proximal cutting member 434. It should be noted that any of the cutting heads described herein may be formed with and located in an outer circumferential wall as shown and described with reference to FIGS. 4A and 4B.

In embodiments, the peripheral wall 43f may be formed separately from the distal cutting member 432 and the proximal cutting member 434. For example, the peripheral wall 43f and the inner catheter body 420 may remain stationary while the distal cutting member 432 and/or the proximal cutting member 434 are rotating. In other embodiments, the peripheral wall 43f is formed integrally with the distal and proximal cutting members 432 and 434 and rotates with the distal and proximal cutting members 432 and 434. The rotatable peripheral wall 43f may provide benefit of increasing turbulence in the vessel during device operation to break up clots. Distal edges 43h of the distal cutting member 432 may provide a cutting edge on its inner edge that can be used during a clot removal operation.

Referring to FIG. 4B, the front-facing cutting head 430 may have a guidewire lumen 438 extending coaxially with an outer catheter lumen 412 and an inner catheter lumen 423 (not shown due to obstruction by other components; shown in FIGS. 6A and 6B). The distal cutting member 432 may extend in a distal direction of the front-facing cutting head 430. The distal cutting member 432 may include one or more blades 43b with cutting edges 43h. The blades 43b may be arranged radially around the shaft 436 of the front-facing cutting head 430 and extend between the peripheral wall 43f and the shaft 436. In the illustrated example, the blades 43b extend integrally from the peripheral wall 43f and the shaft 436 and is a monolithic part of the rest of the front-facing cutting head 430. The blades 43b may be twisted and/or curved to form a thread or a helical-like shape. The blades 43b are spaced-apart from each other to allow fluid to flow between the distal end of the front-facing cutting head 430 and a space 43e formed between the distal cutting member 432, the shaft 436, and the proximal cutting member 434.

The front-facing cutting head 430 may have multiple sets of the blades 43b of the distal cutting member 432 and the blades 43g of the proximal cutting member 434 that are each circumferentially spaced-apart around the shaft 436. The blades 43g may be located proximal of the blades 43b along the longitudinal axis of the front-facing cutting head 430. Like the blades 43b, the blades 43g may be spaced-apart from each other to allow fluid to flow between the space 43e and the inner catheter lumen. The outer catheter lumen 412 is disposed between the outer catheter body 410 and the inner catheter body 420. The outer catheter lumen 412 allows fluid to flow through therein.

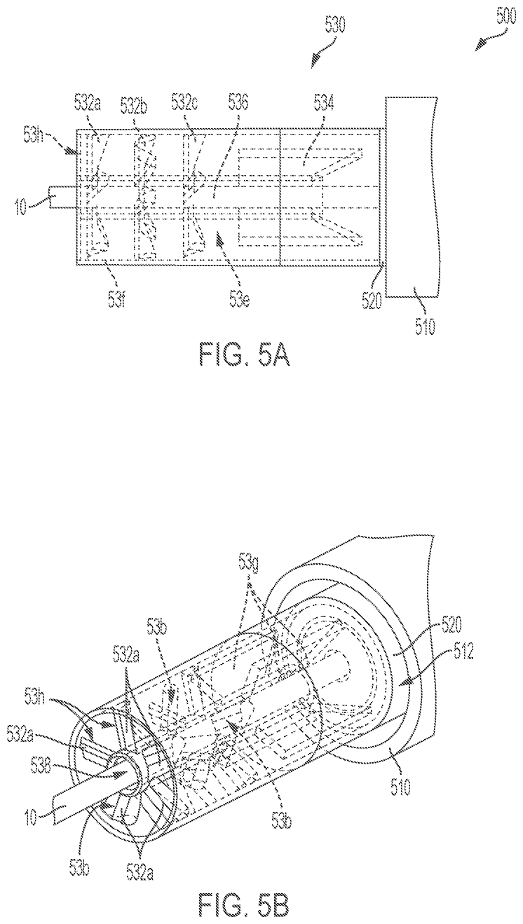

FIGS. 5A and 5B illustrate embodiments of an atherectomy device 500 including a front-facing cutting head 530 with a peripheral wall 53f forming a tube (also represented by element 53f). Referring to FIG. 5A, the front-facing cutting head 530 is in an extended position where the front-facing cutting head 530 is located at least partially distally outside an outer catheter body 510. When in the extended position, the front-facing cutting head 530 and the peripheral wall 53f are uncovered by the outer catheter body 510. The front-facing cutting head 530 may have one or more distal cutting members 532a, 532b, and 532c, a proximal cutting member 534, and a shaft 536 connecting the distal cutting members 532a, 532b, and 532c and the proximal cutting member 534. The front-facing cutting head 530 may have the peripheral wall 53f which may form a tube that radially surrounds one or all of the distal cutting members 532a, 532b, and 532c, the proximal cutting member 534, and the shaft 536. The peripheral wall 53f may be integrally formed with the distal cutting members 532a, 532b, and 532c and/or the proximal cutting member 534 or may be separately formed but fixed to the distal cutting members 532a, 532b, and 532c and/or the proximal cutting member 534. The peripheral wall 53f may rotate together with the distal cutting members 532a, 532b, and 532c and/or the proximal cutting member 534. It should be noted that any of the cutting heads described herein may be formed with and located in an outer circumferential wall as shown and described with reference to FIGS. 5A and 5B. In embodiments of the front-facing cutting head 530, removed tissue passes through the multiple cutting members including one or more distal cutting members 532a, 532b, and 532c, and a proximal cutting member 534, thus “chopping” tissue into a smaller size at successive points in order to reduce the likelihood of blockages in the inner catheter body 520. The multiple cutting member configuration may provide a wider rpm range as the communication of excised tissue through the inner catheter body 520 will be more efficient with smaller pieces.

In embodiments, the peripheral wall 53f may be formed separately from the distal cutting members 532a, 532b, and 532c and the proximal cutting member 534. For example, the peripheral wall 53f and the inner catheter body 520 may remain stationary while the distal cutting members 532a, 532b, and 532c and/or the proximal cutting member 534 are rotating. In other embodiments, the peripheral wall 53f is formed integrally with the distal and proximal cutting members 532a, 532b, 532c, and 534 and rotates with the distal and proximal cutting members 532a, 532b, 532c, and 534. Distal edges 53h of blades 53b of the distal cutting members 532a, 532b, and 532c may provide a cutting edge on its inner edge that can be used during a clot removal operation.

Referring to FIG. 5B, the front-facing cutting head 530 may have a guidewire lumen 538 extending coaxially with an outer catheter lumen 512 and an inner catheter lumen (not shown due to obstruction by other components; equivalent to the inner catheter lumen 123 in FIG. 1). The distal cutting members 532a, 532b, and 532c may extend in a distal direction of the front-facing cutting head 430. Each of the distal cutting members 532a, 532b, and 532c may include one or more blades 53b with cutting edges 53h. The blades may be arranged radially around the shaft 536 and extend between the peripheral wall 53f and the shaft 536. In the illustrated example, the blades 53b extend integrally from the peripheral wall 53f and the shaft 536 and is a monolithic part of the rest of the front-facing cutting head 530. The blades 53b may be twisted and/or curved to form a thread or a helical-like shape. The blades 53b are spaced-apart from each other to allow fluid to flow between the distal end of the front-facing cutting head 530 and a space 53e formed between the distal cutting members 532a, 532b, and 532c, the shaft 536, and the proximal cutting member 534.

In embodiments, the distal cutting members 532a, 532b, and 532c may be disposed along the shaft 536. For example, the distal cutting members 532a, 532b, and 532c may be spaced apart from each other in certain intervals. The distal cutting members 532a, 532b, and 532c may be radially staggered. For example, the distal cutting members 532a, 532b, and 532c are disposed radially shifted at a certain angle.

The proximal cutting member 534 may have a set of blades 53g that are each circumferentially spaced-apart around the shaft 536. The blades 53g may be located proximal of the distal cutting members 532a, 532b, and 532c along the longitudinal axis of the front-facing cutting head 530. The blades 53g may be radially arranged along the longitudinal axis of the front-facing cutting head 530 between the peripheral wall 53f and the shaft 536. The blades 53g may be extended in the longitudinally along the shaft 536. The blades 53g may be spaced-apart from each other to allow fluid to flow between the space 53e and the inner catheter lumen. The outer catheter lumen 512 is disposed between the outer catheter body 510 and the inner catheter body 520. The outer catheter lumen 512 allows fluid to flow through therein. It should be noted that each of the front facing cutting heads 130, 330, 430, and 530 may be used with the atherectomy device of FIG. 1.

Referring to FIGS. 6A-6B, cross sectional views of the atherectomy device 400 of FIGS. 4A and 4B taken along section line 6A-6A are depicted. The atherectomy device 400 may be advanced in a hollow body where materials to be removed are disposed.

FIG. 6A depicts the outer catheter body 410 covering the front-facing cutting head 430 wherein the front-facing cutting head 430 and the inner catheter body 420 are in the retracted position. As discussed hereinabove, the outer catheter body 410 and the inner catheter body 420 may be moved relative to each other in the axial direction of the atherectomy device 400. When the front-facing cutting head 430 and the inner catheter body 420 are in the retracted position, the outer catheter body 410 may be in an extended position. When the front-facing cutting head 430 and the inner catheter body 420 are in the retracted position, flushing fluid (e.g., saline) may be supplied through the flushing lumen 413 defined between the inner catheter body 420 and the outer catheter body 410, and further defined between the front-facing cutting head 430 and the outer catheter body 410. Flushing fluid may be supplied to create flow 30a, 30b in the longitudinal direction of the atherectomy device 400 from proximal end to distal end between the outer catheter body 410 and the inner catheter body 420. Flushing fluid may flow further downstream to create flow 20a, 20b in the longitudinal direction of the atherectomy device 400 from proximal to distal between the outer catheter body 410 and the front-facing cutting head 430.

During an aspiration procedure, a negative pressure may be provided to create flow 40a, 40b and flow 50a, 50b in the longitudinal direction of the atherectomy device 400 from distal end to proximal end between the outer catheter body 410 and the front-facing cutting head 430. A negative pressure is created through the space 43e formed between the distal cutting member 432, the proximal cutting member 434, and the shaft 436 by the rotation of the front-facing cutting head 430 and the rotatable screw member 426. The negative pressure may create the flow 40a, 40b and the flow 50a, 50b. The flow 40a, 40b and the flow 50a, 50b may help transport materials removed from the hollow bodies to inside of the atherectomy device 400. The flow 40a, 40b and the flow 50a, 50b may also aid cutting power of the front-facing cutting head 430 by transport materials toward the distal cutting member 432 and the proximal cutting member 434.

The lateral openings 424a, 424b may be disposed at the distal end portion 421 of the inner catheter body 420. The lateral openings 424a, 424b may be covered by the outer catheter body 410 when the inner catheter body 420 is in the retracted position. The lateral openings 424a, 424b may be formed in the inner catheter body 420 and may be disposed to face each other in a radial direction. The number and size of lateral openings 424a, 424b may be increased or reduced depending on the size of target material, the size of the atherectomy device 400, or the like. A negative pressure is created by rotation of the rotatable screw member 426 and may aid the flow 50a, 50b. The negative pressure may be applied through the inner catheter lumen 423. The rotatable screw member 426 further transport materials down toward the proximal direction. The flow 40a, 40b and the flow 50a, 50b may be closer to the axis of the atherectomy device 400 than the flow 20a, 20b and the flow 30a, 30b.

Referring to FIG. 6B, the outer catheter body 410 may expose the front-facing cutting head 430 and the inner catheter body 420 when the front-facing cutting head 430 and the inner catheter body 420 are in the extended position where the front-facing cutting head 430 extends out from the outer catheter body 410 and/or a tube (also represented by element 410). As discussed hereinabove, the outer catheter body 410 and the inner catheter body 420 may move relative to each other in the axial direction of the atherectomy device 400. When the front-facing cutting head 430 and the inner catheter body 420 are in the extended position, the outer catheter body 410 may be in a retracted position since the outer catheter body 410 moves relative to the front-facing cutting head 430 and the inner catheter body 420. When the front-facing cutting head 430 and the inner catheter body 420 are in the extended position, flushing fluid may be supplied through the flushing lumen 413 defined by the space between the inner catheter body 420 and the outer catheter body 410. Flushing fluid may be supplied to create the flow 30a, 30b in the longitudinal direction of the atherectomy device 400 from proximal to distal between the outer catheter body 410 and the inner catheter body 420. In embodiments where the inner catheter body 420 is movable in an axial direction of the atherectomy device 400 relative to the other of the outer catheter body 410 and the inner catheter body 420 between extended and retracted positions, a user of the atherectomy device 400 may retract the front-facing cutting head 430 when passing the atherectomy device 400 through the heart or other sensitive structures. The atherectomy device 400 may be operated with the front-facing cutting head 430 in the extended position, when the user is confident in the location of the atherectomy device 400 relative to the surrounding vessel and the thrombus and wished to maximize the tissue removal performance of the device.

During an aspiration procedure, a negative pressure may be provided to create flow 40a, 40b and flow 50a, 50b in the longitudinal direction of the atherectomy device 400 from distal to proximal in a vicinity of the front-facing cutting head 430. The rotation of the front-facing cutting head 430 may aid the negative pressure through the space 43e formed between the distal cutting member 432, the proximal cutting member 434, and the shaft 436. The flow 40a, 40b and the flow 50a, 50b may help transport materials removed from the hollow bodies to inside of the atherectomy device 400. The flow 40a, 40b and the flow 50a, 50b may also aid cutting power of the front-facing cutting head 430 by transport materials toward the distal cutting member 432 and the proximal cutting member 434.

The flow 60a, 60b may transport materials toward the lateral openings 424a, 424b. It should be noted that fluid from multiple flows 30a, 30b, 60a, 60b may enter through the lateral openings 424a and 424b so long as removed fibrous material may be carried away from the cutting location and into the inner catheter body 420. The lateral openings 424a, 424b may be disposed at the distal end portion 421 of the inner catheter body 420. The lateral openings 424a, 424b may be uncovered when the inner catheter body 420 is in the extended position. The lateral openings 424a, 424b may be formed in the inner catheter body 420 and may be disposed to face each other in a radial direction. Number of lateral openings 424a, 424b may be increased or reduced depending on the size of target material, the size of the atherectomy device 400, or the like. A negative pressure created by rotation of the rotatable screw member 426 may aid the flow 60a, 60b. The negative pressure may be applied through the inner catheter lumen 423. The rotatable screw member 426 further transports materials down toward the proximal direction. The flow 40a, 40b and the flow 50a, 50b may be closer to the axis of the atherectomy device 400 than the flow 30a, 30b and the flow 60a, 60b.

Referring to FIG. 7, a system, in some embodiments, may include the atherectomy device 100 and a driver 80. The driver 80 may be coupled to the front-facing cutting head 130 to rotate the front-facing cutting head 130. The driver 80 may also be coupled to the rotatable screw member 126 to rotate the rotatable screw member 126. The driver 80 may be a motor (e.g., an electric motor, a hydraulic motor, or the like). The driver 80 may include a driver clutch to provide increased torque for the front-facing cutting head 130 to better remove materials. The system may include an aspiration device 90, such as a syringe, a vacuum syringe, a suction pump, an aspiration pump, or other aspiration source coupled to the atherectomy device 100. The aspiration device 90 may provide a negative pressure to aspirate removed material during an aspiration procedure. The system or the atherectomy device 100 may include a marker (e.g., a marker band or the like) which may aid visibility in conjunction with medical imaging technologies. The system of the atherectomy device 100 may include an external measurement system, such as a catheter shaft marking system which may aid in placement of the atherectomy device 100 without using computerized tomography (CT) scans.

Embodiments can be described with reference to the following numerical clause:
1. An atherectomy device, comprising: an outer catheter body; an inner catheter body that extends through an outer catheter lumen of the outer catheter body; a front-facing cutting head rotatably connected to a distal end of the inner catheter body; and a rotatable screw member extending through an inner catheter lumen of the inner catheter body, wherein the rotatable screw member is configured to carry away material from the front-facing cutting head as the rotatable screw member rotates, wherein a space between the outer catheter body and the inner catheter body defines a flushing lumen and the rotatable screw member having a guidewire lumen that extends coaxially with the outer catheter lumen and the inner catheter lumen.
2. The atherectomy device of any preceding clause, wherein one of the outer catheter body and inner catheter body is movable in an axial direction of the atherectomy device relative to the other of the outer catheter body and inner catheter body between extended and retracted positions.
3. The atherectomy device of any preceding clause, wherein the inner catheter body includes a lateral opening at a distal end portion of the inner catheter body in fluid communication with the inner catheter lumen.
4. The atherectomy device of clause 3, wherein the outer catheter body covers the lateral opening when the outer catheter body is in the extended position and/or the inner catheter body is in the retracted position.
5. The atherectomy device of clause 3 or 4, wherein the outer catheter body uncovers the lateral opening when the outer catheter body is in the retracted position and/or the inner catheter body is in the extended position.
6. The atherectomy device of any preceding clause, wherein the inner catheter lumen defines an aspiration lumen where material is carried away using the rotatable screw member.
7. The atherectomy device of any preceding clause, wherein the front-facing cutting
8. The atherectomy device of any preceding clause, wherein the front-facing cutting head further includes a peripheral wall, and the one or more blades are coupled to the peripheral wall such that the peripheral wall rotates with the one or more blades.
9. The atherectomy device of any of clauses 1 to 7, wherein the front-facing cutting head comprises a peripheral wall being stationary relative to the inner catheter body as the one or more blades rotate.
10. The atherectomy device of any preceding clause, wherein the one or more blades are serrated.
11. The atherectomy device of any preceding clause, wherein the front-facing cutting head is coupled to the rotatable screw member such that the front-facing cutting head and the rotatable screw member are configured to rotate together.
12. The atherectomy device of any preceding clause, wherein the guidewire lumen extends through the front-facing cutting head.
13. A system, comprising: an atherectomy device including: an outer catheter body; an inner catheter body that extends through an outer catheter lumen of the outer catheter body; a front-facing cutting head rotatably connected to a distal end of the inner catheter body; and a rotatable screw member extending through an inner catheter lumen of the inner catheter body, wherein the rotatable screw member is configured to carry away material from the front-facing cutting head as the rotatable screw member rotates; and a driver coupled to the front-facing cutting head to rotate the front-facing cutting head, wherein a space between the outer catheter body and the inner catheter body defines a flushing lumen and the rotatable screw member having a guidewire lumen that extends coaxially with the outer catheter lumen and the inner catheter lumen.
14. The system of clause 13, wherein one of the outer catheter body and inner catheter body is movable in an axial direction of the atherectomy device relative to the other of the outer catheter body and inner catheter body between extended and retracted positions.
15. The system of clause 13 or 14, wherein the inner catheter body includes a lateral opening at a distal end portion of the inner catheter body in fluid communication with the inner catheter lumen.
16. The system of clause 15, wherein the outer catheter body covers the lateral opening when the outer catheter body is in the extended position and/or the inner catheter body is in the retracted position.
17. The system of clause 15 or 16, wherein the outer catheter body uncovers the lateral opening when the outer catheter body is in the retracted position and/or the inner catheter body is in the extended position.
18. A method of using an atherectomy device, comprising: advancing the atherectomy device including: an outer catheter body; an inner catheter body that extends through an outer catheter lumen of the outer catheter body; a front-facing cutting head rotatably connected to a distal end of the inner catheter body; and a rotatable screw member extending through an inner catheter lumen of the inner catheter body, wherein the rotatable screw member is configured to carry away material from the front-facing cutting head as the rotatable screw member rotates; wherein a space between the outer catheter body and the inner catheter body defines a flushing lumen and the rotatable screw member having a guidewire lumen that extends coaxially with the outer catheter lumen and the inner catheter lumen; and rotating the front-facing cutting head.
19. The method of clause 18, further comprising: supplying fluid through the flushing lumen; and applying a negative pressure through the inner catheter lumen.
20. The method of clause 18 or 19, further comprising: moving one of the outer catheter body and the inner catheter body in an axial direction of the atherectomy device relative to the other of the outer catheter body and the inner catheter body to uncover a lateral opening of the inner catheter body, wherein the lateral opening is disposed at a distal end portion of the inner catheter body and is in fluid communication with the inner catheter lumen.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. An atherectomy device, comprising:
an outer catheter body;
an inner catheter body that extends through an outer catheter lumen of the outer catheter body;
a front-facing cutting head having a portion rotatably connected to a distal end of the inner catheter body; and
a rotatable screw member extending through an inner catheter lumen of the inner catheter body, wherein the rotatable screw member is configured to carry away material from the front-facing cutting head as the rotatable screw member rotates,
wherein a space between the outer catheter body and the inner catheter body defines a flushing lumen and the rotatable screw member having a guidewire lumen that extends coaxially with the outer catheter lumen and the inner catheter lumen.

2. The atherectomy device of claim 1, wherein one of the outer catheter body and the inner catheter body is movable in an axial direction of the atherectomy device relative to the other of the outer catheter body and the inner catheter body between extended and retracted positions.

3. The atherectomy device of claim 2, wherein the inner catheter body includes a lateral opening at a distal end portion of the inner catheter body in fluid communication with the inner catheter lumen.

4. The atherectomy device of claim 3, wherein the outer catheter body covers the lateral opening when the outer catheter body is in the extended position and/or the inner catheter body is in the retracted position.

5. The atherectomy device of claim 3, wherein the outer catheter body uncovers the lateral opening when the outer catheter body is in the retracted position and/or the inner catheter body is in the extended position.

6. The atherectomy device of claim 1, wherein the inner catheter lumen defines an aspiration lumen where material is carried away using the rotatable screw member.

7. The atherectomy device of claim 1, wherein the front-facing cutting head includes one or more blades.

8. The atherectomy device of claim 7, wherein the front-facing cutting head further includes a peripheral wall, and the one or more blades are coupled to the peripheral wall such that the peripheral wall rotates with the one or more blades.

9. The atherectomy device of claim 7, wherein the front-facing cutting head comprises a peripheral wall being stationary relative to the inner catheter body as the one or more blades rotate.

10. The atherectomy device of claim 7, wherein the one or more blades are serrated.

11. The atherectomy device of claim 1, wherein the front-facing cutting head is coupled to the rotatable screw member such that the front-facing cutting head and the rotatable screw member are configured to rotate together.

12. The atherectomy device of claim 1, wherein the guidewire lumen extends through the front-facing cutting head.

13. A system, comprising:
an atherectomy device including:
an outer catheter body;
an inner catheter body that extends through an outer catheter lumen of the outer catheter body;
a front-facing cutting head rotatably connected to a distal end of the inner catheter body; and
a rotatable screw member extending through an inner catheter lumen of the inner catheter body, wherein the rotatable screw member is configured to carry away material from the front-facing cutting head as the rotatable screw member rotates; and
a driver coupled to the front-facing cutting head to rotate the front-facing cutting head,
wherein a space between the outer catheter body and the inner catheter body defines a flushing lumen and the rotatable screw member having a guidewire lumen that extends coaxially with the outer catheter lumen and the inner catheter lumen.

14. The system of claim 13, wherein one of the outer catheter body and inner catheter body is movable in an axial direction of the atherectomy device relative to the other of the outer catheter body and inner catheter body between extended and retracted positions.

15. The system of claim 14, wherein the inner catheter body includes a lateral opening at a distal end portion of the inner catheter body in fluid communication with the inner catheter lumen.

16. The system of claim 15, wherein the outer catheter body covers the lateral opening when the outer catheter body is in the extended position and/or the inner catheter body is in the retracted position.

17. The system of claim 15, wherein the outer catheter body uncovers the lateral opening when the outer catheter body is in the retracted position and/or the inner catheter body is in the extended position.

18. A method of using an atherectomy device, comprising:
advancing the atherectomy device including:
an outer catheter body;
an inner catheter body that extends through an outer catheter lumen of the outer catheter body;
a front-facing cutting head rotatably connected to a distal end of the inner catheter body; and
a rotatable screw member extending through an inner catheter lumen of the inner catheter body, wherein the rotatable screw member is configured to carry away material from the front-facing cutting head as the rotatable screw member rotates;
wherein a space between the outer catheter body and the inner catheter body defines a flushing lumen and the rotatable screw member having a guidewire lumen that extends coaxially with the outer catheter lumen and the inner catheter lumen; and rotating the front-facing cutting head.

19. The method of claim 18, further comprising:
supplying fluid through the flushing lumen; and
applying a negative pressure through the inner catheter lumen.

20. The method of claim 18, further comprising:
moving one of the outer catheter body and the inner catheter body in an axial direction of the atherectomy device relative to the other of the outer catheter body and the inner catheter body to uncover a lateral opening of the inner catheter body, wherein the lateral opening is disposed at a distal end portion of the inner catheter body and is in fluid communication with the inner catheter lumen.
